(19) United States
(12) Patent Application Publication
(10) Pub. No.: US 2015/0157806 A1
(43) Pub. Date: Jun. 11, 2015

(54) INJECTION NEEDLE

(71) Applicant: Vigmed AB, Helsingborg (SE)

(72) Inventor: Per Knutsson, Helsingborg (SE)

(21) Appl. No.: 14/412,115

(22) PCT Filed: Jul. 3, 2013
(86) PCT No.: PCT/SE2013/050862

§ 371 Date: Dec. 30, 2014

(30) Foreign Application Priority Data
Jul. 4, 2012 (SE) 1250764-6

(51) Int. Cl. A61M 5/32

(52) U.S. Cl. CPC A61M 5/3213; A61M 5/3293

(57) ABSTRACT

A needle shield for an injection needle assembly is provided. The needle shield comprises a proximal base plate, said base plate having an opening for allowing a needle shaft to run there through, and a distal cover plate, said cover plate having an opening for allowing the needle shaft to run there through. A skin adhesive material is arranged on the distal side of the distal cover plate. An injection needle assembly comprising such a needle shield is also provided.

### CROSS-REFERENCE TO RELATED APPLICATIONS

This application is a U.S. National phase based on PCT/SE2013/050862, filed on Jul. 3, 2013 entitled “INJECTION NEEDLE” which is based on Swedish Patent Application No. 1250764-6, filed on Jul. 4, 2012.

### TECHNICAL FIELD

The present disclosure relates to an injection needle arranged to be shielded from accidental contact.

### BACKGROUND

A pointed hollow needle may be utilized for the administration of solutions or suspensions, such as medicaments, to a human or animal. After puncturing of the skin and introduction of the needle tip, the content of a syringe, typically connected to the rear part of the injection needle, is administered to the human or animal through the hollow injection needle. The injection needle has then done its duty and is withdrawn. An unprotected withdrawn injection needle constitutes, however, a serious health hazard due to the fact that it may be contaminated with e.g. infectious agents originating from the patient's blood or other body fluids, in combination with the needle tip's inherent ability to easily penetrate skin. Hence, the medical personnel who are handling the withdrawn injection needle may acquire the corresponding disease, e.g. HIV or hepatitis, if by accident contacting it with their skin. In order to circumvent or alleviate the health hazards associated with such a withdrawn injection needle amongst other things, there has been much effort devoted to the development of various kinds of semiautomatic injection needle tip protectors.

A traditional device comprises a unit of two pivoted arms of which one is arranged at the syringe hub and the other at the needle tip protector. After injection and withdrawal of the needle from the skin, the user presses the linking part of the unit forwards, whereby the needle tip protector slides towards and clamps the needle tip. However, since the pivoted arms have to be connected to the needle hub, the manufacturing process is quite cumbersome, necessarily comprising multiple manufacturing steps or difficult molding procedures. With these solutions the whole needle hub, safety system, and needle cover have to be specifically manufactured, paying attention to the configuration of the other parts. Additionally, these systems are accompanied by the need for subsequent activation of the needle shield, whereby the needle tip still being hazardously exposed to the user.

Another device comprises a plunger rod which is extending past the needle tip after activation by pushing the plunger to the most forward position, after injection and withdrawal of the needle from the skin. Although such a rod is providing protection when the needle is engaging the skin from a perpendicular direction, there is a risk that the needle may cause dermal wounds if the needle engages the skin close to parallel or at a minor angle towards the skin.

Disadvantages of traditional injection needle tip protectors include their complicated constructions and/or their inherent bulkiness. The former being associated with a large cost of production and the latter with at least an increased volume of bio-hazard waste. In addition, none of above described protectors clamps the needle tip immediately after withdrawal of the needle from the skin. Hence, a user may accidently come in contact with the needle tip in the time span between the withdrawal of the needle and the activation of the needle tip protector by the user.

Hence, an improved injection needle and device for automatic shielding of the needle tip of the injection needle after its employment for injection is desired.

### SUMMARY

Accordingly, the present disclosure seeks to mitigate, alleviate or eliminate one or more of the above-identified deficiencies and to provide an improved needle shield for an injection needle assembly, said needle shield comprising: a proximal base plate, said base plate having an opening for allowing a needle shaft to run there through; and a distal cover plate, said cover plate having an opening for allowing the needle shaft to run there through; wherein a skin adhesive material is arranged on the distal side of the distal cover plate. An injection needle assembly comprising such a needle shield is also provided for the same reasons.

Further features of the disclosure and its embodiments are set forth in the appended claims.

### BRIEF DESCRIPTION OF THE DRAWINGS

These and other aspects, features and advantages of which the disclosure is capable will be apparent and elucidated from the following description of non-limiting embodiments of the present disclosure, reference being made to the accompanying drawings, in which

FIG. 1 is a side view and a cross sectional view of an embodiment of the present disclosure;

FIG. 2 is a cross sectional view of a needle shield according to one embodiment of the present disclosure;

FIG. 3 is a cross sectional view of a needle shield according to one embodiment of the present disclosure;

FIG. 4 is a perspective view of a needle shield according to one embodiment of the present disclosure; and

FIG. 5 is a cross sectional view of a needle shield according to one embodiment of the present disclosure arranged on a needle shaft to cover the needle tip.

### DETAILED DESCRIPTION OF THE EMBODIMENTS

Embodiments of the present invention will be described in more detail below with reference to the accompanying drawings in order for those skilled in the art to be able to carry out the disclosure.

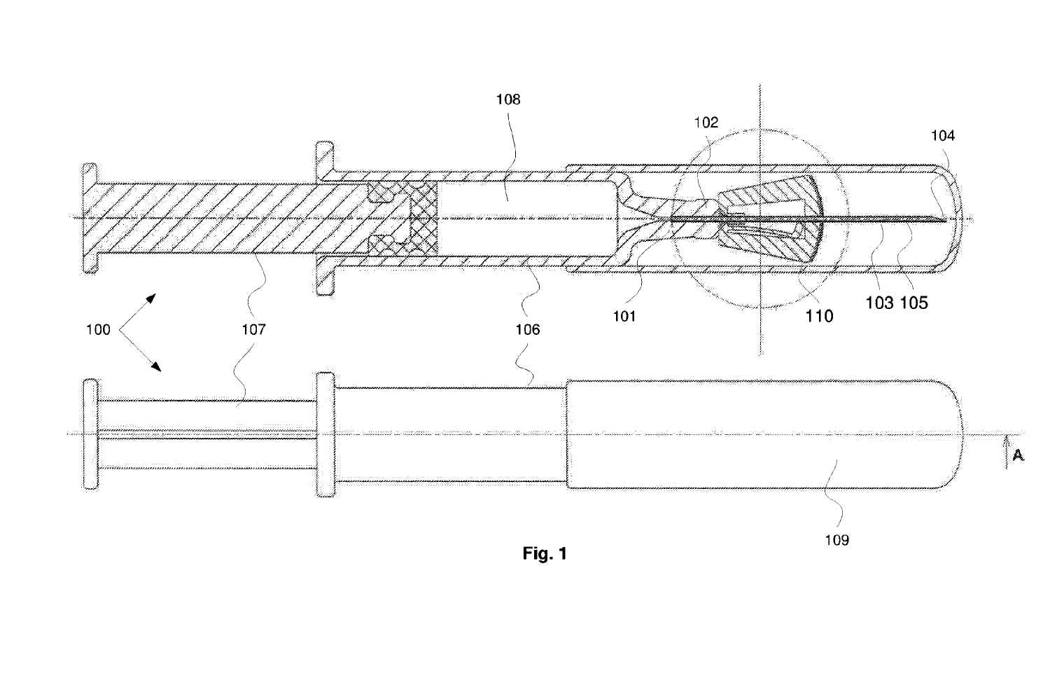

As disclosed in FIG. 1 an injection needle assembly 100 for injecting a fluid into a mammal is disclosed. The injection needle assembly 100 comprises a needle hub 101. The needle hub 101 in turn comprises a proximal needle hub body 102 and a tubular needle shaft 103 with an open distal end and an open proximal end, extending distally from the needle hub body 102. A needle tip 104 is provided in the distal end of the injection needle assembly 100. A bulge 105 is provided on said tubular needle shaft 103 in between said open distal end and said open proximal end. The bulge 105 is provided at the distal end zone of the needle shaft 103. The bulge 105 may be a weld or manufactured by slightly punching the needle shaft 103 at the intended location of the bulge 105.

Proximally of the needle hub 101 a syringe 106 is provided. The needle hub 101 may be integrated with the syringe 106, but it may also be provided as a separate part, which is attachable via a luer lok or luer slip to the distal top of the syringe 106. The syringe 106 comprises a plunger 107, running in a syringe cylinder 108, such that the plunger 107 may push a liquid contained in the syringe 106 out through the distal top end of the syringe 106 into the lumen of the needle shaft 103, in a manner known to the skilled artisan.

Onto the needle hub 101, and over the needle shaft 103 and needle tip 104, a cylindrical cover 109 may or may not be applied, depending on the circumstances.

Distally of the syringe 106, a needle shield 110 is applied onto the needle shaft 103. The needle shield 110 may be of a plastic material, such as polycarbonate. When the needle shield 110 is of a plastic material, the entire needle shield 110 may be injection molded into one monolithic piece. The needle shield 110 is slidable along the longitudinal axis of the needle shaft 103, between a first proximal inactive position, wherein the proximal part of the needle shield 110 sits on the distal end of the syringe 106, and a distal active position, wherein the needle shield 110 covers the needle tip 104 and is prohibited from proximal displacement to again uncover the needle tip 104.

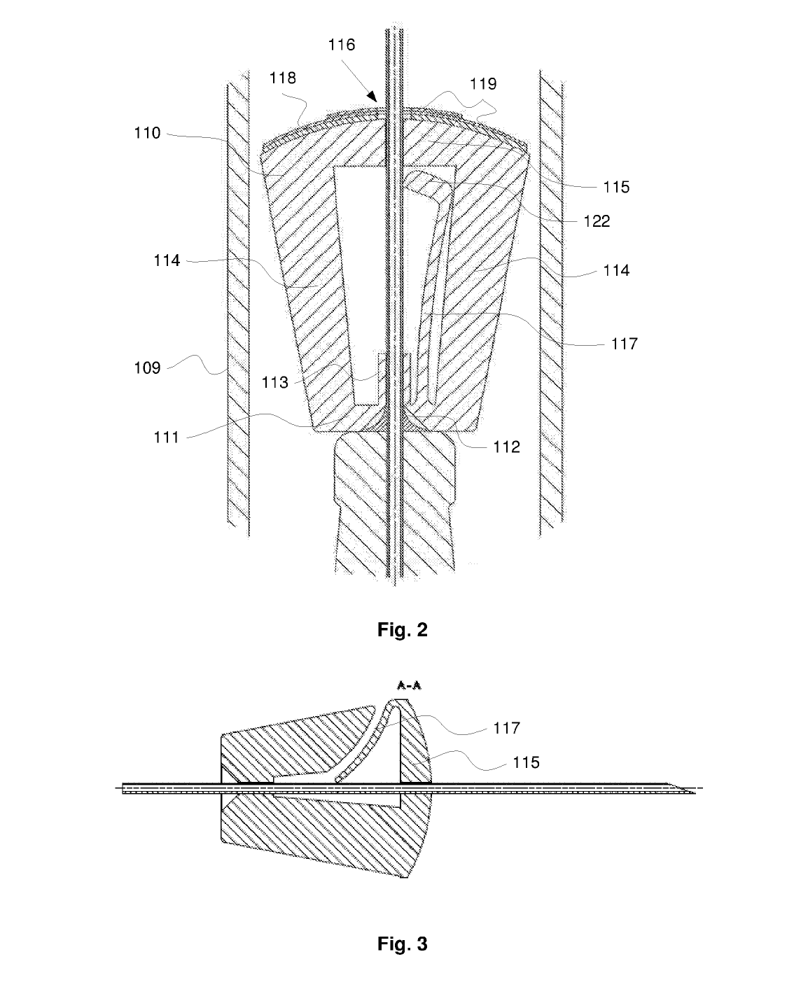

The needle shield 110 comprises a proximal base plate 111, as illustrated in FIG. 2. The base plate 111 is configured to sit on the distal end of the syringe 106. This may be accomplished via a central cavity 112 at the proximal end surface of the base plate 111. The central cavity 112 is adapted to receive the so frequently present conical connection between the needle shaft 103 and the needle hub 101 or syringe 106. The central cavity also aids insertion of the needle in a needle shaft seat 113, since it will guide the needle tip into the needle shaft seat 113. The needle shaft seat 113 is arranged in the base plate 111, such that the needle shaft 103 may pass proximodistally there through. The needle shaft seat 113 may have a tubular lumen, or it may be tube-shaped. When the needle shaft seat 113 is tube-shaped, the outside thereof may be slightly conical, for allowing better releasing properties during molding. The tubular lumen of the needle shaft seat 113 will stabilize the needle. When the needle shaft seat 113 is of a plastic material, it is possible to obtain a tubular needle shaft seat 113, which improves the stabilization of the needle shaft 103 running there through, such that other stabilizing means for making sure that the needle shaft 103 not wobbles or sways in a way jeopardizing the function of the needle shield may be avoided. From the base plate 111, laterally from the needle shaft seat 113, beams 114 extend distally. The beams 114 may have a I or H shaped cross section, to stabilize the beams 114 from unwanted bending or torsion, which could set the needle tip 104 free from the needle shield 110. At the distal end of the beams 114, a distal cover plate 115 is positioned. The distal cover plate 115 is provided with a through opening 116, extending at least in the proximodistal direction, for allowing a needle shaft 103 running through the needle shaft seat 113 to also pass through the opening 116. The opening 116 may be a slit extending centrally from the periphery of the cover plate 115. To provide the opening 116 as a slit from the periphery of the cover plate 115 facilitates manufacturing, since injection molding is largely facilitated by omitting unnecessary injection molding cores. The cover plate 115 could be of a suitable shape for allowing good contact characteristics, such as flat or spherical. The cross sectional shape is preferably circular, to maximize contact area and to facilitate manufacturing.

Distally of the shaft seat 113 and proximally of the cover plate 115, a flexible arm 117 may be arranged. The flexible arm 117 may extend distally from the base plate 111 and rest upon the needle shaft 103 at the distal end of the flexible arm in the inactive state. The flexible arm 117 may however also extend from the cover plate 115, as disclosed in FIG. 3, or the beams 114, as long as it may urged from a resting state by the needle shaft 103 when the needle shield is in an inactive position into a loaded state, such that it can return to said resting state to cover the needle tip 104 when the needle tip 104 passes proximally beyond the distal end of the flexible arm 117. The needle shield 110 may be injection molded such that the flexible arm 117 has a resting state, wherein the distal end of the flexible end intersects a longitudinal axis through the tubular shaft seat 113, such that the flexible arm 117 will be urged into said resting state when the needle shaft has been displaced proximally through the needle shield 110 to the position when the needle tip 104 passes proximally beyond the distal end of the flexible arm 117. In this way the flexible arm 117 will cover the needle tip 104, once the needle has been adequately withdrawn into the needle shield 110.

The flexible arm 117 may also be arranged centrally of and behind the beams 114, such that manipulation of the flexible arm 117 is hampered.

On the front side of the cover plate 115, i.e. the side facing distally, the cover plate 115 is provided with a skin adhesive material. The skin adhesive material may be provided as a skin adhesive layer or coating 118. The skin adhesive material, such as the skin adhesive layer or coating 118, may be arranged on the cover plate 115 such that an interspace is formed between the edge of the opening 116 and the skin adhesive material. In this way, contact between the needle shaft 103 and the skin adhesive material may be avoided.

The skin adhesive material of the skin adhesive layer or coating 118 may for example be a pressure sensitive adhesive (PSA) or a curing adhesive (CA). When the skin adhesive material is PSA, the skin adhesive material gives immediate tack while allowing for a smaller interspace between the skin adhesive material and the edge of the opening 116, since the viscosity of PSA is relatively high. Suitable PSA's may be selected from the group comprising hydrocolloids or thermoplastics with suitable resins or rosins as tackifiers, acrylics and acrylates, polyvinylpyrrolidone based, vinyl polymer, silicone based, and latex based with tackifier. When acrylics and acrylates are used, the toxicological properties of the skin adhesive material may be kept within borders for medical use. Suitable CA's may be selected from the group comprising cyanoacrylates or polyvinyl types.

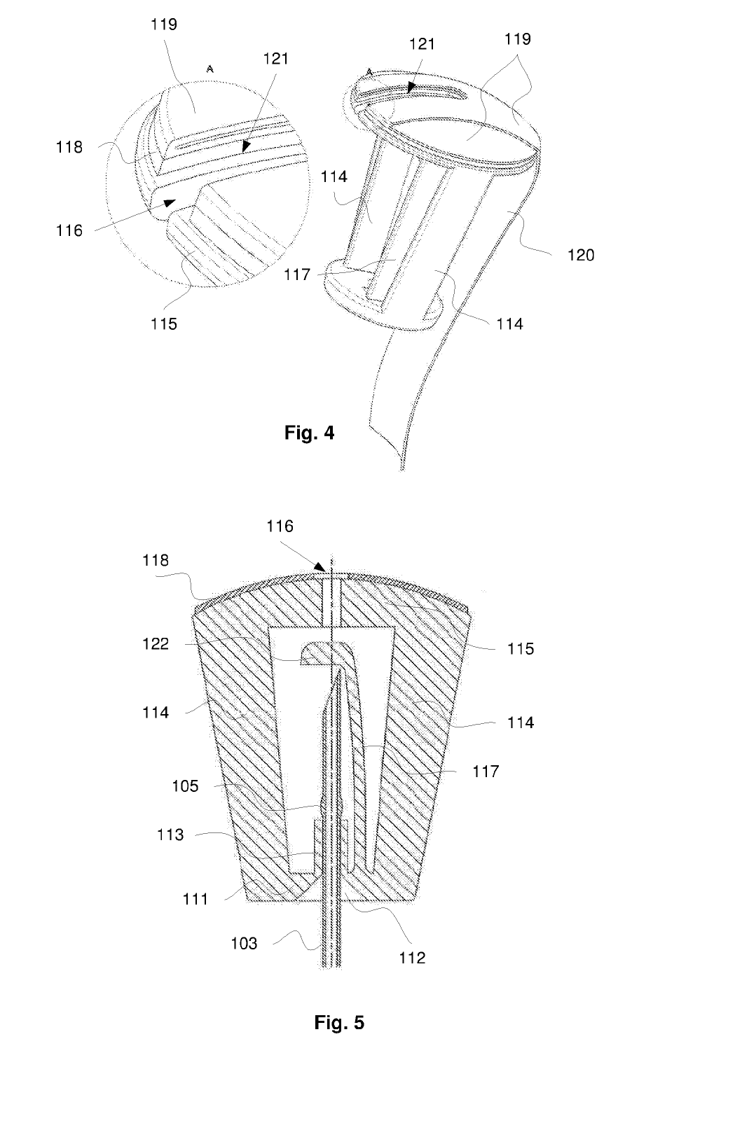

Distally of the skin adhesive material a liner 119 is arranged, as illustrated in FIG. 2 and also in FIG. 4. The liner 119 comprises a pulling tap 120. The liner 119 is arranged in a double folded manner onto the skin adhesive material, and the pulling tap 120 extends from the upper, most distally arranged, sheet of the liner 120, such that when the pulling tap 119 is pulled, the liner 119 will start to be separated from the skin adhesive material on the diagonally from where the pulling tap 120 is arranged. Also, the liner 119 is provided with a through slit 121, extending from the central end of the opening 116 and diagonally away from the pulling tap 120. Due to the double fold and the slit 121, the liner 119 may be easily removed from the skin adhesive material while the needle shaft 103 extends through the cover plate 115 and distally there from, without having to lift the pulling tap above the distally located needle tip 104, when the needle shield 110 is in the inactive state.

In use, the cover 109 is removed by the user, such as a nurse, where after the syringe cylinder 108 is filled with medicament by pulling the plunger 107 proximally or the syringe 106 is prefilled. Thereafter, the user may disinfect the area on the patient where the needle stick will take place. The liner 119 is then removed by pulling the pulling tap radially outward/laterally and perhaps downward/proximally. The liner will then be continuously removed from the skin adhesive material from the diagonal end of the cover plate 115 over the cover plate 115, without interacting with the needle shaft 103. The user then penetrates the skin of the patient with the needle tip 104 and inserts the needle shaft 103 until the skin adhesive material on the cover plate 115 contacts and tacks to the skin of the patient. It is also possible to first inserting the needle into the patient, and thereafter pushing the needle shield forward until the skin adhesive cover plate 115 contacts the skin. When the skin adhesive material contacts the skin of the patient, the skin adhesive material and thus the needle shield 110 attaches to the skin. In this position, it does not matter if the patient will twitch due to the needle stick, bearing in mind that approximately 20% of accidental needle sticks occur during injection, since a twitch from the patient only will result in an immediate cover of the needle tip, if the needle is withdrawn from the patient. The plunger 107 is the pushed distally, to inject the medicament into the patient. Thereafter, the syringe 106 is withdrawn proximally, such that the needle shaft 103 is withdrawn from the patient.

When the needle shaft 103 is withdrawn from the patient, the needle shaft simultaneously moves proximally in relation to the needle shield 110. Thus, the needle shaft 103 travels proximally through the tubular shaft seat 113, until the bulge 105 hits the shaft seat 113, as disclosed in FIG. 5. When the bulge 105 hits the shaft seat 113, the withdrawing force of the syringe 106 overcomes the adherence force between the skin and the skin adhesive material and thus the needle shield 110, whereby the needle assembly 100 again is released from the patient. Just before the bulge 105 hits the shaft seat 113, the needle tip 104 passes proximally beyond the distal end of the flexible arm 117, whereby the flexible arm moves into its memory and relaxed state, in which the flexible arm covers the needle tip 104 and prohibits the needle tip 104 to again be pushed out through the opening 116. The flexible arm 117 may be provided with a hooked distal end 122, to facilitate hindrance of distal needle movement once the needle tip 104 has passed proximally beyond the distal end of the flexible arm 117.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

1. A needle shield for an injection needle assembly, said needle shield comprising:
a proximal base plate, said base plate having an opening for allowing a needle shaft to run there through; and
a distal cover plate, said cover plate having an opening for allowing the needle shaft to run there through;
wherein a skin adhesive material is arranged on the distal side of the distal cover plate.

2. The needle shield according to claim 1, wherein the needle shield is a monolithic piece of plastic material.

3. The needle shield according to claim 2, wherein the plastic material is polycarbonate.

4. The needle shield according to claim 1, wherein the opening in the base plate is a tubular needle shaft seat.

5. The needle shield according to claim 1, wherein at least one beam, such as two beams extend distally from the base plate, and that the cover plate is connected to the distal end of the at least one beam.

6. The needle shield according to claim 5, wherein the at least one beam, such as two beams, have a I or H shaped cross section.

7. The needle shield according to claim 1, wherein the opening in the distal cover plate is a slit extending centrally from the periphery of the cover plate.

8. The needle shield according to claim 1, wherein the cover plate is flat or spherical, with a circular cross sectional shape.

9. The needle shield according to claim 1, wherein a flexible arm is arranged distally of the base plate and proximally of the cover plate.

10. The needle shield according to claim 9, wherein the flexible arm extends distally from the base plate, said flexible arm in a resting position intersecting a central axis of the opening in the base plate, and having a flexibility such that it may be urged by a needle shaft to not intersect with said central axis.

11. The needle shield according to claim 9, wherein the flexible arm extends proximally from the cover plate, said flexible arm in a resting position intersecting a central axis of the opening in the base plate, and having a flexibility such that it may be urged by a needle shaft to not intersect with said central axis.

12. The needle shield according to claim 9, wherein the flexible arm extends from the at least one beam, said flexible arm in a resting position intersecting a central axis of the opening in the base plate, and having a flexibility such that it may be urged by a needle shaft to not intersect with said central axis.

13. The needle shield according to claim 9, wherein the flexible arm is at least one of positioned centrally and behind the beams.

14. The needle shield according to claim 1, wherein the skin adhesive material is selected from the group consisting of hydrocolloids or thermoplastics with suitable resins or rosins as tackifiers, acrylics and acrylates, polyvinylpyrrolidone based, vinyl polymer, silicone based, and latex based with tackifier.

15. The needle shield according to claim 1, wherein a liner is arranged distally of the skin adhesive material.

16. The needle shield according to claim 15, wherein the liner comprises a pulling tap.

17. The needle shield according to claim 15, wherein the liner is arranged in a double folded manner onto the skin adhesive material.

18. The needle shield according to claim 17, wherein the liner is provided with a through slit, extending from the central end of the opening.

19. An injection needle assembly for injecting a fluid into a mammal, said injection needle assembly comprising:
a needle hub, said needle hub comprising a needle hub body and a tubular needle shaft with a distal end needle tip, said needle shaft having a bilge at its distal zone;
a needle shield arranged onto said needle shaft, such that the needle shaft passes through the opening in the base plate and the opening in the cover plate.

20. The injection needle assembly according to claim 19, wherein a syringe is at least one of the needle hub and provided proximally of the needle hub.
